(19) United States
(12) Patent Application Publication
(10) Pub. No.: US 2020/0291368 A1
(43) Pub. Date: Sep. 17, 2020

(54) Improved CRISPR-Cpf1 Genome Editing Tool

(71) Applicants: Wageningen Universiteit, Wageningen (NL); Erasmus University Medical Center Rotterdam, Rotterdam (NL)

(72) Inventors: John VAN DER OOST, Renkum (NL); Peter VAN BAARLEN, Wageningen (NL); Rogier Petrus Leonardus LOUWEN, Rotterdam (NL)

(21) Appl. No.: 16/084,011

(22) PCT Filed: Mar. 10, 2017
(86) PCT No.: PCT/NL2017/050153

§ 371 Date: Sep. 11, 2018

(30) Foreign Application Priority Data
Mar. 11, 2016 (EP) 16160010.1

(51) Int. Cl. C12N 9/22; C12N 15/11; C12N 15/88

(52) U.S. Cl. CPC C12N 9/22; C12N 15/11; C12N 2310/3513; C12N 2310/20; C12N 2800/80; C12N 15/88

(57) ABSTRACT

The invention relates to a Cpf1-based nuclease complex, wherein the guide RNA sequence is irreversibly crosslinked to the Cpf1 protein. The cross-link may be a covalent binding or a non-covalent binding. Such a complex may be used in delivering constructs to a cell that are capable of gene-editing. Use of this cross-linked complex will result in less off-targeting.

The invention relates to the field of genetics, more particular to the field of gene editing, especially gene editing through the CRISPR-Cpf1 system.

CRISPR sequences are Clustered Regularly Interspaced Short Palindromic Repeat sequences that are present in bacteria and archaea. Initially these kind of sequences have been indicated as Short Regularly Spaced Repeats (SRSRs) (Mojica, F. J. et al., 2000, Mol. Microbiol. 36:244-246), but they have been renamed in the acronym CRISPR by Jansen et al. (Jansen, R. et al., 2002, Mol. Microbiol. 43:1565-1575). Their function has been revealed later by Doudna and Charpentier, who independently from each other showed that CRISPR sequences work together with proteins from the Cas (CRISPR associated) group to form a kind of immune reaction against viral infections (Pennisi, E., 2013, Science 341:833-836). Recently, it has been found by the groups of Koonin, Van der Oost and Zhang that CRISPR sequences can also work together with a different enzyme, Cpf1 (Zetsche, B. et al., 2015, Cell 163:1-13).

In this article it is shown that Cpf1, like Cas9, one of the Cas enzymes, in connection with an adapted version of the CRISPR (guide) sequences could be used for genetic editing. In the past years the CRISPR-Cas system has been studied extensively and currently it is one of the most promising tools in genetic engineering because of its ease of use (e.g. Young, S. 2014, MIT Technol. Review: http://www.technologyreview.com/review/524451/genome-surgery/; Mali, P. et al., 2013, Nature Meth. 10:957-963). However, it is proposed that the CRISPR-Cpf1 system may outperform the CRISPR-Cas9 system.

Cpf1 (CRISPR from Prevotella and Francisella (Zetsche et al., 2015)) is an RNA-guided DNA endonuclease enzyme. Cpf1 is a class 2 CRISPR effector, which may perform without a tracrRNA and which utilizes a 5′ T-rich protospacer-adjacent motif (PAM). Cpf1 cleaves DNA via a staggered DNA double-stranded break.

The Cpf1 enzyme has been isolated from the bacteria Francisella novicida. The Cpf1 protein contains a predicted RuvC-like endonuclease domain that is distantly related to the respective nuclease domain of Cas9. However, Cpf1 differs from Cas9 in that it lacks HNH, a second endonuclease domain that is present within the RuvC-like domain of Cas9. Furthermore, the N-terminal portion of Cpf1 is predicted to adopt a mixed a/8 structure and appears to be unrelated to the N-terminal, α-helical recognition lobe of Cas9 (Makarova, K. et al., Nat. Rev. Microbiol. 13:722-736, 2015; Shmakov, S. et al., Mol. Cell. 60:385-397, 2015).

One disadvantage of the CRISPR-Cas9 system is that in many cases off-targeting mutagenesis occurs during this gene-editing. This off-targeting is thought to be caused by nonspecific interaction of the guide RNA and the target DNA, and/or by malfunctioning of the Cas9 enzyme. Although less is known about the off-targeting when Cpf1 is used instead of Cas9, it is thought that off-targeting also occurs with Cpf1 and that it would be advantageous to have embodiments for gene editing where off-targeting will occur less. However, some recent structure-inspired protein engineering studies revealed significantly improved specificity of Cas9 (Slaymaker, I. et al., Science 351:84-88, 2016); it is anticipated that this can also be achieved for Cpf1.

One approach to mitigate off-target activity has centered on the optimalisation of choice of the guide RNA. The last few years have seen a rapid development of software packages to aid in the guide RNA design process by undertaking exhaustive target sequence searches against genomic reference sequences, allowing the selection of target sequences with minimal off-target cleavage effects (Naito et al., 2015, Bioinformatics 31:1120-1123). However, this merely enables efficient exploration of the target sequence space available for guide sequence design rather than directly addressing the inherent limitations of CRISPR-Cas9 (i.e., dependence on a specific guide RNA and a second RNA molecule, a tracrRNA) as a genome editing tool.

The present inventors now found that a solution to the problem of preventing off-targeting can be provided by using a Cpf1-based nuclease complex, wherein the guide RNA sequence is irreversibly crosslinked to the Cpf1 protein. Preferably in said complex the guide RNA sequence comprises a CRISPR nucleic acid sequence. It is further preferred that the guide RNA is not derived from the same organism as the Cpf1 protein.

In said complex the Cpf1 protein preferably is derived from Acidominococcus and Lachnospiraceae, and more preferably is derived from Francisella novicida, Porphyromonas macacae, Prevotella disiens, Porphyromonas crevioricanis, Lachnospiraceae bacterium ND2006, Lachnospiraceae bacterium MC2017, Leptospira inadai, Moraxella bovoculi 237, Eubacterium eligens, Candidatus Methanoplasma termitum, Methanomethylophylus alvus, Butyrivibrio proteoclasticus, Smithella sp. SC_K08D17, Lachnospiraceae bacterium MA2020, or Acidaminococcus sp. BV3L). Most preferred is the embodiment wherein the Cpf1 enzyme is derived from Francisella novicida, preferably from Francisella novicida U112.

Also part of the invention is a complex as described above wherein the guide RNA is coupled to the Cpf1 enzyme through an RNA linker molecule. A preferred embodiment of the present invention is a complex as described above wherein the guide RNA is covalently coupled to the Cpf1 protein, preferably wherein the covalent coupling is established by UV irradiation. In this embodiment the coupling is preferably made via the backbone of the RNA molecule.

In an alternative embodiment the guide RNA is non-covalently complexed with the Cpf1 protein.

Further part of the invention is a method for delivering a construct capable of gene editing to a eukaryotic cell, comprising the steps of:
a. providing a complex as described above; and
b. introducing said eukaryotic cell with said vector.

Further part of the invention is a method for gene editing a eukaryotic cell comprising providing a complex as described above to said cell. Preferably in such a method of the invention said cell is part of an organism, preferably wherein the organism is selected from the group of fungi, algae, plants and animals, including human.

Further included in the invention is the use of a cross-linked complex of Cpf1 and a guide RNA for gene-editing, preferably gene-editing of eukaryotic cells.

### DETAILED DESCRIPTION

### Definitions

All technical and scientific terms used herein have the same meaning as commonly understood by one of ordinary skill in the art to which this invention belongs, unless the technical or scientific term is defined differently herein.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

“Genomic DNA” refers to the DNA of a genome of an organism including, but not limited to, the DNA of the genome of a bacterium, fungus, archaea, plant or animal.

“Manipulating” DNA encompasses binding, nicking one strand, or cleaving (i.e., cutting) both strands of the DNA, or encompasses modifying the DNA or a polypeptide associated with the DNA (e.g., amidation, methylation, etc.). Manipulating DNA can silence, activate, or modulate (either increase or decrease) the expression of an RNA or polypeptide encoded by the DNA.

“Gene-editing” refers to the process of changing the genetic information present in the genome of a cell. This gene-editing may be performed by manipulating genomic DNA, resulting in a modification of the genetic information. Such gene-editing may or may not influence expression of the DNA that has been edited.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e. not include any mismatches.

By “hybridizable” or “complementary” or “substantially complementary” it is meant that a nucleic acid (e.g. RNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 55%, at least 60%, at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need to be sequence-specific (e.g., contacts with phosphate residues in a DNA backbone), but some portions of a binding interaction may be sequence-specific. Binding interactions are generally characterized by a dissociation constant (Kd) of less than 10⁻⁶M, less than 10⁻⁷M, less than 10⁻⁸M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd. By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.) and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide is “homologous” to, or has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including http://blast.ncbi.nlm.nih.gov/Blast.cgi?CMD=Web&PAGE_TYPE=BlastDocs &DOC_TYPE=Download. See, e.g., Altschul et al. (1990), J. Mol. Biol. 215:403-10. Sequence alignments standard in the art are used according to the invention to determine amino acid residues in a Cpf1 ortholog that “correspond to” amino acid residues in another Cpf1 ortholog. The amino acid residues of Cpf1 orthologs that correspond to amino acid residues of other Cpf1 orthologs appear at the same position in alignments of the sequences.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence a transcription initiation site will be found, as well as protein binding domains responsible for the binding of RNA polymerase. Eukaryotic promoters will often, but not always, contain so-called “TATA” boxes and “CAT” boxes. Various promoters, including inducible promoters, may be used to drive the vectors as described in the present disclosure.

A promoter can be a constitutively active promoter (i.e., a promoter that is constitutively in an active (“ON”) state), it may be an inducible promoter (i.e., a promoter whose state, active (“ON”) or inactive (“OFF”), is controlled by an external stimulus, e.g., the presence of a particular temperature, compound, or protein). It may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hibernation in plants).

In principle the same promoters may be used for expressing the Cpf1 enzyme as the promoters that are used for expressing the Cas enzymes. Where in the prior art suitable promoters for the expression of the Cas proteins have been derived from viruses (which can therefore be referred to as viral promoters) also preferred are the bacterial promoters that are used to express the Cas9 protein in wild-type bacteria. It is also possible that a promoter that is known to drive Cas9 expression in one bacterium is used to drive the expression of a Cas9 or Cpf1 protein derived from a different (species) of bacterium. In such case, it is said that said promoter is heterologous with respect to the Cas9 or Cpf1 protein. Alternatively, they can be derived from any organism, including prokaryotic or eukaryotic organisms. Suitable promoters can be used to drive expression by any RNA polymerase (e.g., in prokaryotes bacterial RNA polymerase, T7 RNA polymerase, and in eukaryotes pol I, pol II, pol Ill). Exemplary viral promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a Rous sarcoma virus (RSV) promoter. Human promoters comprise a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

Examples of inducible promoters include, but are not limited to T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSEN02, X51956); an aromatic amino acid decarboxylase (MDC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med.16(10): 1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKIM) promoter (see, e.g., Mayford et al. (1996) Proc. Natl. Acad. Sci. USA 93: 13250; and Casanova et al. (2001) Genesis 31:37); a CMV enhancer/platelet-derived growth factor-p promoter (see, e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25: 1476; and Sato et al. (2002) J. Biol. Chem. 277: 15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262: 187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (see, e.g., Akyiirek et al. (2000) Mol. Med. 6:983; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9: 1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-protein-coding sequence (e.g., guide RNA) or a protein-coding sequence (e.g., Cpf1 polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to two components that are defined by structures derived from different sources. For example, where “chimeric” is used in the context of a chimeric polypeptide (e.g., a chimeric Cpf1 protein), the chimeric polypeptide includes amino acid sequences that are derived from different polypeptides. A chimeric polypeptide may comprise either modified or naturally-occurring polypeptide sequences (e.g., a first amino acid sequence from a modified or unmodified Cpf1 protein; and a second amino acid sequence other than the Cpf1 protein). Similarly, “chimeric” in the context of a polynucleotide encoding a chimeric polypeptide includes nucleotide sequences derived from different coding regions (e.g., a first nucleotide sequence encoding a modified or unmodified Cpf1 protein; and a second nucleotide sequence having another function, such as a nuclear localization signal).

The term “chimeric polypeptide” refers to a polypeptide which is not naturally occurring, e.g., is made by the artificial combination (i.e., “fusion”) of two otherwise separated segments of amino sequence through human intervention. A polypeptide that comprises a chimeric amino acid sequence is a chimeric polypeptide. Some chimeric polypeptides can be referred to as “fusion variants.”

“Heterologous,” as used herein, means a nucleotide or peptide that is not found in the native nucleic acid or protein, respectively. For example, in a chimeric Cpf1 protein, the RNA-binding domain of a naturally-occurring bacterial Cpf1 polypeptide (or a variant thereof) may be fused to a heterologous polypeptide sequence (i.e. a polypeptide sequence from a protein other than Cpf1 or a polypeptide sequence from another organism). The heterologous polypeptide may exhibit an activity (e.g., enzymatic activity) that will also be exhibited by the chimeric Cpf1 protein (e.g., nuclease activity, methyltransferase activity, acetyltransferase activity, kinase activity, etc.). A heterologous nucleic acid may be linked to a naturally-occurring nucleic acid (or a variant thereof) (e.g., by genetic engineering) to generate a chimeric polynucleotide encoding a chimeric polypeptide. As another example, in a fusion variant Cpf1 site-directed polypeptide, a variant Cpf1 site-directed polypeptide may be fused to a heterologous polypeptide (i.e. a polypeptide other than Cpf1), which exhibits an activity that will also be exhibited by the fusion variant Cpf1 site-directed polypeptide. A heterologous nucleic acid may be linked to a variant Cpf1 site-directed polypeptide (e.g., by genetic engineering) to generate a polynucleotide encoding a fusion variant Cpf1 site-directed polypeptide. “Heterologous,” as used herein, additionally means a nucleotide or polypeptide in a cell that is not its native cell.

In a preferred embodiment, a fusion variant is fused with a restriction endonuclease or a modified restriction endonuclease, e.g. FokI. Sharkey is a high-activity FokI nuclease domain (Guo, J. et al., J. Mol. Biol. 400:96-107, 2010). Such a modification preferably occurs in the catalytic domain, such as are available from the KKR Sharkey or ELD Sharkey proteins which may be fused to the Cpf1 protein. KKR and ELD refer to specific amino acid substitutions in the two binding domains of a dimeric FokI protein. In a preferred application of these complexes of the invention, two of these complexes (KKR Sharkey and ELD Sharkey) may be combined. A pair of protein complexes (heterodimer) which comprises differently modified FokI enzymes has particular advantage in targeted double stranded cutting of nucleic acid. If homodimers are used then it is possible that there is more cleavage at non-target sites due to non-specific activity.

A heterodimer approach advantageously increases the fidelity of the cleavage in a sample of material.

The term “cognate” refers to two biomolecules that normally interact or co-exist in nature.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence.

Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

The term “recombinant bacteria” means bacteria which have been modified by a change in the total nucleic acid content which is contained in such bacteria. This change can be effected by introduction of a heterologous nucleic acid, but it may also be effected by a non-naturally induced change in the nucleic acid, such as a mutation, wherein this mutation can comprises a replacement of nucleic acids, an insertion of nucleic acids or a deletion of nucleic acids.

An “expression vector” is a replicon, such as plasmid, phage, virus, or cosmid, to which another DNA segment, i.e. an “insert”, may be attached so as to bring about the replication of the attached segment in a cell. A “vector” in the present application is an organism, such as viruses or bacteria that may be used to transfer nucleic acids, proteins and/or bacteria into another organism. Especially in the present invention a vector is used to transfer the crosslinked Cas9-DNA complex into the target cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. Alternatively, a cell has been “genetically modified” or “transformed” or “transfected” by introducing into said cell a crosslinked Cpf1-DNA complex as defined in the present application.

In prokaryotes, yeast, and mammalian cells for example, the transforming DNA may be maintained on an episomal element such as a plasmid. With respect to eukaryotic cells, a stably transformed cell is one in which the transforming DNA or (a part of) the DNA part that is present on the Cpf1-DNA complex has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PE1)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. p ii: 50169-409X(12)00283-9. doi: 10.1016/j.addr.2012.09.023), and the like.

Numerous transfection methods have been developed to transfer proteins and other macromolecules across the plasma membrane efficiently. These include physical methods, such as electroporation, sonoporation, cell squeezing, magnetofection, optical transfection, impalefection and microinjection, as well as a chemical or biological carrier-mediated methods. Chemical transfection reagents such as cationic lipids or polymers are widely used, either alone or in combination with scaffolds. Biological methods include delivery with cell-penetrating protein domain fusions such as trans-activator of transcription protein from human immunodeficiency virus, VP22 or Antennapedia peptides. Certain proteins such as zinc-finger nucleases, which are used for targeted genome modification such as the abovementioned FokI dimer, even appear to have intrinsic cell-penetrating properties. For the specific transfer of the Cpf1-DNA complexes of the present invention transfer systems that are known for proteins may be advantageously used. Such systems comprise penetrating peptides (Wagstaff et al., Curr Med Chem. 2006; 13:1371-1387; Mae et al., Curr Opin Pharmacol. 2006; 6:509-514 and e.g. HSV-VP22 as described by Xiong et al., BMC Neuroscience 2007, 8:50), some of which may be commercially available (e.g. the Xfect™ Protein Transfection Reagent from Clontech), proteoliposomes (Liguouri L., Meth Enzymol. 2009; 465:209-223) and lipid based transfection systems (e.g. the Fuse-it-P™ system from Ibidi, Planegg, Germany; PULSin® from Source Bioscience, Nottingham, UK)) and viral based vesicle systems, such as from Vaccinia virus (Temchura et al., 2008 Jul. 4; 26(29-30):3662-72), capsids from polyoma virus (Bertling, W., Buiosci. Rep. 1987, 7(2):107-112) or by virus-derived nanovesicles VSV-G induced nanovesicles as described in Mangeot et al., Molecular Therapy (2011) 19 9, 1656-1666).

The choice of method of genetic modification is generally dependent on the type of cell being transformed and the circumstances under which the transformation or transfection is taking place (e.g., in vitro, ex vivo, or in vivo). A general discussion of these methods can be found in Ausubel, et al., Short Protocols in Molecular Biology, 3rd ed., Wiley & Sons, 1995.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed or transfected by the nucleic acid or a complex with said nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid or a complex with such a nucleic acid (e.g., a plasmid, vector or recombinant expression vector) and a eukaryotic host cell is a genetically modified eukaryotic host cell (e.g., a mammalian germ cell), by virtue of introduction into a suitable eukaryotic host cell of an exogenous nucleic acid or a complex with such a nucleic acid.

A “target DNA” as used herein is a DNA polynucleotide that comprises a “target site” or “target sequence.” The terms “target site.” “target sequence,” “target protospacer DNA.” or “protospacer-like sequence” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “non-complementary strand” or “non-complementary strand.” By “site-directed modifying polypeptide” or “RNA-binding site-directed polypeptide” or “RNA-binding site-directed modifying polypeptide” or “site-directed polypeptide” it is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA. thus targeting the bound polypeptide to a specific location within the target DNA (the target sequence).

By “cleavage” is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a site-directed modifying polypeptide is used for targeted double-stranded DNA cleavage.

“Nuclease” and “endonuclease” are used interchangeably herein to mean an enzyme which possesses endonucleolytic catalytic activity for DNA cleavage.

By “cleavage domain” or “active domain” or “nuclease domain” of a nuclease it is meant the polypeptide sequence or domain within the nuclease enzyme which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The RNA molecule that binds to the site-directed modifying polypeptide and targets the polypeptide to a specific location within the target DNA is referred to herein as the “guide RNA” or “guide RNA polynucleotide” (also referred to herein as a “guide RNA” or “gRNA”). A guide RNA comprises two segments, a “DNA-targeting segment” and a “CRISPR repeat segment.” By “segment” it is meant a segment/section/region of a molecule, e.g., a contiguous stretch of nucleotides in an RNA. A segment can also mean a region/section of a complex such that a segment may comprise regions of more than one molecule. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules. However, the total length of the RNA molecule should at least be 32 nucleotides, having at least 16-17 nucleotides for the DNA targeting segment and at least 16, but optimally more than 17 nucleotides for the direct repeat segment.

The DNA-targeting segment (or “DNA-targeting sequence”) comprises a nucleotide sequence that is complementary to a specific sequence within a target DNA (the complementary strand of the target DNA) designated the “protospacer-like” sequence herein. The protein-binding segment (or “protein-binding sequence”) interacts with a site-directed modifying polypeptide. For Cpf1 site-specific cleavage of the target DNA occurs at locations determined by both (i) base-pairing complementarity between the guide RNA and the target DNA; and (ii) a short motif (referred to as the protospacer adjacent motif (PAM)) in the target DNA. This PAM motif for Cpf1 is the sequence ‘(T)TTN’, where N may be any nucleotide. Remarkably, and in contrast to what is known from the Cas9 nuclease, the PAM motif for Cpf1 is located at the 5′ side of the target DNA. Although closely related 5′ T-rich PAMs have been revealed for 8 Cpf1 variants (Zetsche, 2015), orthologs of Cpf1 may need the same, related or different PAM sequence on the target.

The direct repeat of a guide RNA comprises, in part, two complementary stretches of nucleotides that hybridize to one another to form a double stranded RNA duplex (stem loop structure). It is believed that the Cpf1 nuclease recognizes the direct repeat segment (for this reason also indicated as peptide-binding segment) on basis of a combination of sequence-specific and structural features of the stem loop and adjacent sequences.

In some embodiments, a nucleic acid (e.g., a guide RNA, a nucleic acid comprising a nucleotide sequence encoding a guide RNA; a nucleic acid encoding a nuclease; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like), more specifically a modification or sequence that provides a binding site for cross-linking it to a nuclease enzyme; and combinations thereof.

In some embodiments, a guide RNA comprises an additional segment at either the 5′ or 3′ end that provides for any of the features described above. For example, a suitable further segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) more specifically a modification or sequence that provides a binding site for cross-linking it to a nuclease enzyme; and combinations thereof.

A guide RNA and a nuclease form a complex (i.e., bind via non-covalent interactions). The guide RNA provides target specificity to the complex by comprising a nucleotide sequence that is complementary to a sequence of a target DNA. The nuclease of the complex provides the site-specific activity. In other words, the nuclease is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding (direct repeat) segment of the guide RNA.

As indicated above, a major difference with the Cas9 system concerns the fact that Cpf1 does not use a tracrRNA for processing, and subsequently for anchoring the mature crRNA guide to the effector protein. Instead, Cpf1 has a single RNA, with a stem-loop structure at the 5′ end that may function as a site for RNA processing, and for anchoring to the Cpf1 effector protein. In most embodiments, a guide RNA comprises only one separate RNA segment, the so-called “targeter-RNA”, see below) and is referred to herein as a “single-molecule guide RNA”, “smRNA, or a “one-molecule guide RNA”. The term “guide RNA” or “gRNA” is generally meant to indicate single-molecule guide RNAs (i.e., smRNAs). Such a smRNA is specifically known to be used with the Cpf1 nuclease (Zetsche et al., Cell, 2015). The Cpf1 related guide RNA comprises a crRNA-like (“CRISPR RNA” or “targeter-RNA”) molecule (which includes a CRISPR repeat or CRISPR repeat-like sequence) and a segment that is able to hybridize to the target DNA.

This single molecule RNA is in contrast to the well-known Cas9 system, which requires a guide RNA having two molecules, a targeter molecule and an activator RNA, also indicated as tracrRNA. Such a two-molecule guide RNA comprises two separate RNA molecules (a “targeter-RNA” and an “activator-RNA”). Each of the two RNA molecules of a two-molecule guide RNA comprises a stretch of nucleotides that are complementary to one another such that the complementary nucleotides of the two RNA molecules hybridize to form the double stranded RNA duplex of the protein-binding segment. An exemplary two-molecule guide RNA comprises a crRNA-like (“CRISPR RNA” or “targeter-RNA”) molecule (which includes a CRISPR repeat or CRISPR repeat-like sequence) and a corresponding tracrRNA-like (“trans-activating CRISPR RNA” or “activator-RNA” or “tracrRNA”) molecule. A crRNA-like molecule (targeter-RNA) comprises both the DNA-targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator-RNA) comprises a stretch of nucleotides (duplex-forming segment) that forms the other half of the dsRNA duplex of the protein-binding segment of the guide RNA In other words, a stretch of nucleotides of a crRNA-like molecule are complementary to and hybridize with a stretch of nucleotides of a tracrRNA-like molecule to form the dsRNA duplex of the protein-binding domain of the guide RNA As such, each crRNA-like molecule can be said to have a corresponding tracrRNA-like molecule. The crRNA-like molecule additionally provides the single stranded DNA-targeting segment. Thus, a crRNA-like and a tracrRNA-like molecule (as a corresponding pair) hybridize to form a guide RNA. A double-molecule guide RNA can comprise any corresponding crRNA and tracrRNA pair.

The term “activator-RNA” is used herein to mean a tracrRNA-like molecule of a double-molecule guide RNA. The term “targeter-RNA” is used herein to mean a crRNA-like molecule of a double-molecule guide RNA or a single molecule RNA. The term “duplex-forming segment” is used herein to mean the stretch of nucleotides of an activator-RNA or a targeter-RNA that contributes to the formation of the dsRNA duplex by hybridizing to a stretch of nucleotides of a corresponding activator-RNA or targeter-RNA molecule. In other words, an activator-RNA comprises a duplex-forming segment that is complementary to the duplex-forming segment of the corresponding targeter-RNA. Therefore, a double-molecule guide RNA can be comprised of any corresponding activator-RNA and targeter-RNA pair.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA In some embodiments, the donor polynucleotide, a portion of the donor polynucleotide, a copy of the donor polynucleotide, or a portion of a copy of the donor polynucleotide integrates into the target DNA

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a plant or an animal, such as a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” and “patient,” are used interchangeably herein and refer to any plant or animal, such as a mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The phrase “consisting essentially of” is meant herein to exclude anything that is not the specified active component or components of a system, or that is not the specified active portion or portions of a molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As has been stated in the introduction, the gene editing of eukaryotic cells has been advanced by the invention that the CRISPR-Cas and the improved CRISPR-Cpf1 system could be used as a target- and replace-mechanism for making mutations in de nucleic acid of the target cell.

The current inventors now have found that a lot of unwanted side effects occur when the nuclease and the guide RNA are present as separate entities in the transformed or transfected cell. Apparently (see the below experimental evidence) the Cas9 enzyme (which has thus far been abundantly used and tested) is steered towards unintended off-target nucleic acid sequences, which may occur when the guide RNA acts aspecifically with respect to the target DNA. Another possibility is that the nuclease recognizes or works together with pieces of RNA/DNA that are already present in the target cell and which thus would play the role of guide RNA/DNA, thereby ‘mis’guiding the nuclease to introduce unintended breaks into protein-coding DNA regions, with harmful consequences to the targeted host cell. It is believed that this is true for all CRISPR based gene editing systems, also in particular for other class 2 CRISPR effectors including the Cpf1 enzyme. It has been shown that mutating the Streptococcus pyogenes Cas9 enzyme can lead to a reduction in this off-targeting effect (Kleinstiver et al., Nature, 2016 doi:10.1038/nature16526). However, in cases in which other enzymes than Cas9 from Streptococcus pyogenes are desirable, such as when using the Cpf1 nuclease other solutions should be sought. One of the possibilities is to prevent the nuclease to complex with other RNAs than the guide RNA with which it is desired to complex.

Such an effect may be achieved by overexpression of the gRNA with respect to (expression of) the nuclease. This can be achieved by providing the cell with a construct in which the gRNA is expressed under control of a strong constitutive promoter, while the nuclease enzyme is expressed in a less abundant number or provided to the cell through transfection. However, since the nuclease and the gRNA will need time to find each other in the cell and form a complex, there still is the risk that off-target effects can occur from unbound nuclease enzyme.

One way to improve on this is by providing the nuclease-guide RNA complex to the cell in a complete manner. In this embodiment, the enzyme and the gRNA are complexed outside the cell, any unbound enzyme then preferably is removed from the solution, and the complexes are then transfected into the cell, e.g. by lipofection. In this case the presence of free nuclease enzyme or nuclease enzyme coupled to pieces of DNA that are endogenous to the cell to be transfected is minimalized.

However, in this embodiment it is still possible that nuclease enzyme and gDNA are separated and that free nuclease can be introduced into the cell where it can exert the deleterious effects. For this reason it is preferred that the nuclease is tightly connected to the guide RNA before the complex would enter the target cell. In the alternative, the prevention of the above-mentioned off-targeting may also be caused by assuring that the nuclease is complexed to the (correct) guide RNA before it exercises its function. A first possible embodiment in which this can be effected is first cross-linking the guide RNA with the nuclease in a cell or in vitro system in which both components are available.

It is preferred that the guide RNA will be cross-linked to the Cpf1 protein or otherwise firmly attached to it to enable proper functioning of the Cpf1-guide RNA complex. Such a cross-linking can be established by means known to the skilled person for crosslinking proteins to nucleic acids. One specific embodiment of such a cross-linking has been described by Saito and Matsura (1985, Acc. Chem. Res. 18:134-141) who described photoreactions with lysine and tryptophan to e.g. thymidine moieties on the nucleic acid.

Such a coupling may be performed by irradiation with UV light. UV crosslinking is irreversible, very specific and only crosslinks protein to RNA; no protein-protein crosslinking does occur (Brimacombe, T R. Et al., Meth. Enzymol. 164:287-309, 1988). It has been established (as shown in a co-pending application) with relation to Cas9, that such a UV crosslinking yields a still functional nuclease. Further, appropriate photo-cross-linking with p-benzoyl-L-phenylalanine (pBpa) has been described (Farrell, I. et al., Nature Meth. 2:377-384, 2005). With the use of this technique the protein to be cross-linked, in this case the Cpf1 enzyme, can be specifically provided with the pBpa at any site of the protein. Also, such a specific cross-linkable site provides for an excellent specificity for the photo-induced cross-linking reaction. Another possibility is to cross-link the nuclease protein with the RNA in a reaction using formaldehyde (Möller, K. et al., 1977, Eur. J. Biochem. 76:175-187). Other bifunctional reagents that may be employed in cross-linking reactions are diepoxybutan (Skold, 1981, Biochimie 63(1):53-60), trans-diaminedichloroplatinum (II) (Tukalo et al., 1987, Biochem. 26(16):5200-5208) and 1-ethyl-3(3-dimethylaminopropyl)carbothimide (EDC). It would also be possible to first modify the RNA molecule (e.g. to contain adenosine moieties modified to contain a disulfide bond and an alkyl chain, where the disulfide bond can be reduced with a reactive thiol and then cross-linked to an amino acid via reaction with benzophenone. Also nucleosides with tethers ending in primary amines (which are e.g. commercially available) can be derivatized with cross-linking reagents containing isothiocyanate or succinimide ester functional groups. A nucleotide that is readily available for cross-linking is 4-thiouridine.

Also, in order to further avoid off-targeting it is preferred that the nuclease used is heterologous to the organism or cell which is targeted by the Cpf1-guide RNA complex. A heterologous Cpf1 enzyme can be provided by transforming an intended bacterial vector or cell with a Cpf1 enzyme from a different source, i.e. different microbial species. Cpf1 orthologous enzymes are known in both bacteria and archaea, and Cpf1 enzymes for the present invention can be predominantly derived from Acidominococcus and Lachnospiraceae, and preferably are derived from Porphyromonas macacae, Prevotella disiens, Porphyromonas crevioricanis, Lachnospiraceae bacterium ND2006, Lachnospiraceae bacterium MC2017, Leptospira inadai, Moraxella bovoculi 237, Eubacterium eligens, Candidatus Methanoplasma termitum, Methanomethylophylus alvus, Butyrivibrio proteoclasticus, Smithella sp., SC_K08D17Lachnospiraceae bacterium MA2020, and Acidaminococcus sp. BV3L). Most preferred is the Cpf1 enzyme from Francisella novicida, preferably from the Francisella novicida U112 strain (Zetsche, B. et al., 2015, Cell, 163(3):759-771). A further advantage of the Cpf1 nuclease is that it does not require a tracrRNA (activator RNA). It processes crRNA arrays without needing tracrRNA and Cpf1-crRNA cuts target DNA without needing any other RNA types. Shorter oligos are easier to deliver and cost less to synthesize. This is particularly useful when chemically modifying single molecule RNAs, which was recently shown to enhance efficiency in human primary cells (Hendel, A. et al., 2015, Nature Biotechnol. 33:985-989). Another advantage is that CRISPR-Cpf1 may be used in situations where the surroundings of the target sequence that is to be cut is very AT rich. Where (the most used) Cas9 enzymes have a PAM sequence containing NGG, the PAM sequence for the Cpf1 enzyme is (T)TTN.

A further difference between Cpf1 and Cas9 is that Cpf1 produces a cleavage with a 5′ overhang, where Cas9 produces cuts with blunt ends. Since the exact cleavage site in the target is known and thereby the sequence of the 5′ overhang, an insert can be genetically engineered to fit with the overhang, which then also would result in a fixed orientation of the inserted segment of nucleotides. In other words, it will be more easy with Cpf1 produced cuts to use single-stranded donor oligonucleotides as source for insertion, since these may hybridize directly to the site of cleavage and do not need the long segments of templates to anneal with the target.

In many cases, i.e. with many Cas9 enzymes it is needed to insert a heterologous NLS signal with the construct that encodes for the Cas9 enzyme to construct a chimeric Cas9 enzyme which would be capable of targeting the nucleus/nucleolus of the cell in order to effect its action. A similar NLS signal for targeting of Cpf1 to the nucleus is also required and preferably two NLS signals will be used for a more efficient functioning of the enzyme.

Preferably, the guide RNA that is used in the present invention comprises a CRISPR sequence (crRNA). Such a CRISPR sequence may be a CRISPR sequence that is derived from bacterial or archaeal origin, but it may also be a CRISPR that is derived from other organisms, such as the CRISPR sequences disclosed in co-pending application PCT/NL2015/050438. As indicated above, for Cpf1 the guide RNA only needs to comprise a DNA-targeting sequence as defined above and possibly a sequence recognizing the protospacer adjacent motif (PAM) sequence. It does not need to contain an activator RNA (tracrRNA) nor the region that would be needed for binding with the activator RNA as is required for Cas9 based guide RNAs.

Cross-linking of the nuclease with the guide RNA may be accomplished in a vector cell, especially the vector cell in which the heterologous nuclease has been expressed and in which also the guide RNA is expressed either on the same construct as the nuclease or by any other means. Cross-linking may also take place in vitro, in a composition comprising the nuclease, where the nuclease is isolated from a cell that produces said nuclease, or as a readily available preparation from a commercial source. Then the guide RNA is added to the composition and cross-linking is performed, such as cross-linking through UV radiation or chemical reaction.

If cross-linking is performed in a living cell or biological vector (such as a virus particle) then advantageously it should be established that as little as possible and preferably no unbound nuclease is left in said cell or vector. This can be advantageously accomplished by starting the cross-linking reaction with a higher concentration of guide RNA. Excess guide RNA will not lead to off-targeting effects.

If cross-linking is performed in vitro the cross-linked complex may be taken up by a vector for delivering it to a target cell. The cross-linked complex may also be directly delivered into a target cell by any of the known transfection methods, such as electroporation, iTOP (Neijssen, J. et al., Nature 434:83-88, 2005) and the like. It is also possible to use systems or methods that are known to be able to transfer macromolecules into a cell, such as liposomes and/or cationic amphiphilic compounds.

The Cpf1 enzyme exhibits nuclease activity that cleaves target DNA at a target DNA sequence defined by the region of complementarity between the guide RNA and the target DNA. Then site-specific cleavage of the target DNA occurs at locations determined by both (i) base-pairing complementarity between the guide RNA and the target DNA; and (ii) a short motif [referred to as the protospacer adjacent motif (PAM)] in the target DNA. Cpf1 proteins from various species, such as the species listed above may require different PAM sequences in the target DNA. Thus, for a particular Cpf1 protein of choice, the PAM sequence requirement may be different than the 5′-TTN-3′ sequence described above.

The nuclease activity cleaves target DNA to produce double strand breaks, but with an overhang of the 5′ end. These breaks are then repaired by the cell in one of two ways: non-homologous end joining, and homology-directed repair. In non-homologous end joining (NHEJ), the double-strand breaks are repaired by deletion of the overhanging nucleotides followed by direct ligation of the blunt ends to one another. As such, no new nucleic acid material is inserted into the site, but some nucleic acid material will be lost, resulting in a deletion. In homology-directed repair, a donor polynucleotide with homology to the cleaved target DNA sequence is used as a template for repair of the cleaved target DNA sequence, resulting in the transfer of genetic information from the donor polynucleotide to the target DNA. This can be done by direct homology to the overhanging nucleotides that result from the cleavage, but it may also be accomplished by introducing a new nucleic acid material with homology to the target sequence a bit further upside or downside from the break. As such, new nucleic acid material may be inserted/copied into the site. In some cases, a target DNA is contacted with a donor polynucleotide. In some cases, a donor polynucleotide is introduced into a cell. The modifications of the target DNA due to NHEJ and/or homology-directed repair lead to, for example, gene correction, gene replacement, gene tagging, transgene insertion, nucleotide deletion, gene disruption, gene mutation, sequence replacement, etc.

In some embodiments, the Cpf1 protein comprises a heterologous sequence which can provide for subcellular localization of the site-directed modifying polypeptide (e.g., a nuclear localization signal (NLS) for targeting to the nucleus; a mitochondrial localization signal for targeting to the mitochondria; a chloroplast localization signal for targeting to a chloroplast; a ER retention signal; and the like). In some embodiments, a heterologous sequence can provide a tag for ease of tracking or purification (e.g., a fluorescent protein, e.g., green fluorescent protein (GFP), YFP, RFP, CFP, mCherry, tdTomato, and the like; a his tag, e.g., a 6×His tag; a hemagglutinin (HA) tag; a FLAG tag; a Myc tag; and the like). In some embodiments, the heterologous sequence can provide for increased or decreased stability.

The Cpf1 gene can be codon-optimized. This type of optimization is known in the art and entails the mutation of foreign-derived DNA to mimic the codon preferences of the intended vector or host organism or cell while encoding the same protein. Thus, the codons are changed, but the encoded protein remains unchanged. For example, if the intended target cell was a human cell, a human codon-optimized Cpf1 (or variant, e.g., fusion protein) would be a suitable enzyme. While codon optimization is not required, it is acceptable and may be preferable in certain cases. Polyadenylation signals can also be chosen to optimize expression in the intended host.

In some of the above applications, the methods may be employed to induce DNA cleavage, DNA modification, and/or transcriptional modulation in mitotic or post-mitotic cells in vivo and/or ex vivo and/or in vitro (e.g., to produce genetically modified cells that can be reintroduced into an individual).

Because the guide RNA provides specificity by hybridizing to target DNA, a target cell of interest in the disclosed methods may include a cell from any eukaryotic organism (e.g. a cell of a single-cell eukaryotic organism, a plant cell, an algal cell, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a primate, a cell from a human, etc.).

Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Human embryo's and cells derived from human embryos are excluded from the present invention. Cells may be from established cell lines or they may be primary cells, where “primary cells”, “primary cell lines”, and “primary cultures” are used interchangeably herein to refer to cells and cells cultures that have been derived from a and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. Target cells are in many embodiments unicellular organisms, or are grown in culture.

If the cells are primary cells, they may be harvested from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

In some embodiments, a method involves contacting a target DNA or introducing into a cell (or a population of cells) the cross-linked complex of a guide RNA and a Cpf1 enzyme and/or a donor polynucleotide.

Contacting the cells with a cross-linked complex of a guide RNA and Cpf1 enzyme and/or donor polynucleotide may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive.

Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Conditions that promote the survival of cells are typically permissive of non-homologous end joining and homology-directed repair. In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by the Cpf1 enzyme. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide. The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signal expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequence differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. Such methods have also been used successfully to protect guide RNAs from degradation (Hendel, A. et al., Nat. Biotechnol. 33:985-989, 2015). As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by bacterial or viral vectors, as described above for nucleic acids encoding a guide RNA and/or nuclease and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5,385,582); by bolus injection, e.g. by a syringe, e.g. into a joint; by continuous infusion, e.g. by cannulation, e.g. with convection (see e.g. US Application No. 20070254842); or by implanting a device upon which the cells have been reversibly affixed (see e.g. US Application Nos. 20080081064 and 20090196903). Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the disclosure, the guide RNA and/or nuclease and/or donor polynucleotide are employed to modify cellular DNA in vivo, again for purposes such as gene therapy, e.g. to treat a disease or as an antiviral, antipathogenic, or anticancer therapeutic, for the production of genetically modified organisms in agriculture, or for biological research. In these in vivo embodiments, the Cpf1 enzyme cross-linked to a guide RNA and/or a donor polynucleotide are administered directly to the individual. When these components are administered in addition to the administration through a vector as described herein before, they may be administered by any of a number of well-known methods in the art for the administration of peptides, small molecules and nucleic acids to a subject. Such a peptide or nucleic acid component can be incorporated into a variety of formulations. More particularly, the guide RNA—nuclease complex and/or donor polynucleotide can be formulated into pharmaceutical compositions by combination with appropriate pharmaceutically acceptable carriers or diluents.

Accordingly, the gene-editing method as discussed above may be used to delete nucleic acid material from a target DNA sequence, to remove disease-causing trinucleotide repeat sequences in neurons, to create gene knockouts and mutations as disease models in research, etc. by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material into a locus of choice in the target DNA.

Alternatively, if the cross-linked complex of a guide RNA and a Cpf1 enzyme is co-administered to cells with a donor polynucleotide sequence that includes at least a segment with homology to the target DNA sequence, e.g. by providing all components within one and the same vector or administration vehicle, the subject methods may be used to add, i.e. insert or replace, nucleic acid material to a target DNA sequence (e.g. to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, a complex comprising a guide RNA and a Cpf1 enzyme is useful in any in vitro or in vivo application in which it is desirable to modify DNA in a site-specific, i.e. “targeted”, way, for example gene knock-out, gene knock-in, gene editing, gene tagging, sequence replacement, etc., as used in, for example, gene therapy, e.g. to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

1. A Cpf1-based nuclease complex comprising a Cpf1 protein and a guide RNA sequence, wherein the guide RNA sequence is irreversibly crosslinked to the Cpf1 protein.

2. The complex according to claim 1, wherein the guide RNA sequence comprises a CRISPR nucleic acid sequence.

3. The complex according to claim 1,
wherein the guide RNA is not derived from the same organism as the Cpf1 protein-.

4. The complex according to claim 1, wherein the Cpf1 protein is derived from Acidominococcus and Lachnospiraceae, and preferably is derived from Francisella novicida, Porphyromonas macacae, Prevotella disiens, Porphyromonas crevioricanis, Lachnospiraceae bacterium ND2006, Lachnospiraceae bacterium MC2017, Leptospira inadai, Moraxella bovoculi 237, Eubacterium eligens, Candidatus Methanoplasma termitum, Methanomethylophylus alvus, Butyrivibrio proteoclasticus, Smithella sp. SC_K08D1 7, Lachnospiraceae bacterium MA2020, and Acidaminococcus sp. BV3L).

5. The complex according to claim 4, wherein the Cpf1 enzyme is derived from Francisella novicida.

6. The complex according to claim 1, wherein the guide RNA is coupled to the Cpf1 enzyme through an RNA linker molecule.

7. The complex according to claim 1 wherein the guide RNA is covalently coupled to the Cpf1 protein.

8. The complex according to claim 7, wherein the covalent coupling is established by UV irradiation.

9. The complex according to claim 8, wherein the coupling is made via the backbone of the RNA molecule.

10. The complex according to claim 1, wherein the guide RNA is non-covalently complexed with the Cpf1 protein.

11. Method for delivering a construct capable of gene editing to a eukaryotic cell, said cell not being a human germ-line cell, comprising the steps of:
a. providing a construct comprising a complex according to claim 1; and
b. introducing said construct into said eukaryotic cell.

12. Method for gene editing a eukaryotic cell comprising providing a complex according to claim 1 to said cell.

13. Method according to claim 11 or 12, wherein said cell is part of an organism, preferably wherein the organism is selected from the group of fungi, algae, plants and animals, including human.

14. (canceled)

15. Method for gene editing a eukaryotic cell comprising providing a complex between a Cpf1 protein and a guide RNA and introducing said complex into the cell.

16. Method according to claim 15, wherein said introduction into the cell is performed by lipofection.

17. Method for gene editing a eukaryotic cell comprising providing a construct encoding a Cpf1 protein and a construct encoding a guide RNA, wherein the guide RNA is overexpressed with respect to the Cpf1 protein by being expressed under control of a strong promoter.

18. The complex according to claim 5, wherein the Cpf1 enzyme is derived from Francisella novicida U112.
